(19) United States
(12) Patent Application Publication
(10) Pub. No.: US 2002/0022224 A1
(43) Pub. Date: Feb. 21, 2002

(54) Method for isolating single-stranded DNA

(76) Inventors: David P. Hornby, Widnes (GB); Mark Dickman, Broomhill (GB)

Correspondence Address: WILLIAM B. WALKER, TRANSGENOMIC, INC., 2032 CONCOURSE DRIVE, SAN JOSE, CA, 95131, US

(21) Appl. No.: 09/770,846

(22) Filed: Jan. 26, 2001

Related U.S. Application Data
(60) Provisional application No. 60/200,824, filed on Apr. 28, 2000.
(60) Provisional application No. 60/221,340, filed on Jul. 26, 2000.

(51) Int. Cl. C12Q 1/68; C12P 19/34

(52) U.S. Cl. 435/6; 435/91.1

(57) ABSTRACT

A method for generating single stranded DNA (ssDNA) directly from double stranded PCR (dsPCR) products is described. The method generally entails: (1) amplifying a target polynucleotide by means of two oligonucleotide primers, wherein one primer is capable of hybridizing to the target polynucleotide and the other primer is capable of hybridizing to the complement of the target polynucleotide, and wherein one of the primers comprises a chemical tag, thereby producing an amplification product mixture comprising a tagged amplification product of the target polynucleotide and a complementary non-tagged amplification product; (2) applying the amplification product mixture to a separation medium, wherein the chemical tag is capable of interacting with the separation medium; and (3) eluting the amplification products from the separation medium by means of a mobile phase under denaturing conditions, wherein the interaction between the tag and the separation medium results in the physical separation of the two amplification products.

### CROSS REFERENCE TO RELATED CO-PENDING APPLICATIONS

This application claims priority to U.S. Provisional Application No. 60/200,824, filed Apr. 28, 2000 and U.S. Provisional Application No. 60/221,340, filed Jul. 26, 2000, both of which are incorporated by reference herein in their entirety.

### FIELD OF THE INVENTION

The present invention is directed to the separation and purification of poynucleotides.

### BACKGROUND OF THE INVENTION

Single stranded DNA (ssDNA) is an extremely important reagent in molecular biology. The purification and isolation of ssDNA is a key step in numerous analytical molecular biology procedures including strand specific hybridization (Pagratis and Revel (1990) Virology 177:273-80), in vitro selection of ssDNA aptamers (Bock et al. (1992) Nature 355:564-66; Ellington and Szostak (1992) Nature 355:850-52), nucleotide sequencing (Sambrook et al, (1989) in Molecular Cloning: A Laboratory Manual Cold Spring Harbor Lab. Plainview, N.Y.) and molecular weight analysis of DNA using mass spectrometetry (Limbach et al. (1995) Curr. Opin. Biotechnol. 6:96-102). A variety of methods have been used previously to obtain ssDNA from double stranded (ds) PCR products, including asymmetric PCR (Kaltenboeck et al. (1992) Biotechniques 12:164-71), amplification of DNA with modified primers that result in the synthesis of products of unequal length (Williams and Bartel (1995) Nucleic Acids Res. 23:4220-21), asymmetric exonucleolytic digestion of a double stranded PCR product (Higuchi & Ochman (1989) Nucleic Acids Res. 17:5865; Hannon et al. (1993) Anal. Biochem. 212:421-27), affinity purification procedures (Hultman et al. (1995) Nucleic Acids Res. 17:4937-46; Mitchell & Merril (1989) Anal. Biochem. 178:239-42; and Green et al. (1990) Nucleic Acids Res. 18:6163-64) and more recently gel retardation techniques (Pagratis (1996) Nucleic Acids Res. 24:3645-46). None of these methods generate highly purified sinlge stranded DNA in high yields. Furthermore, these methods are often either time consuming or do not allow the size dependent purification and analysis of the single stranded DNA product. Affinity purification procedures offer a rapid means of isolating ssDNA; however, downstream analysis is required to monitor purification efficiency. Importantly, most of the techniques described above require more than one step to isolate the purified ssDNA from dsPCR products.

The instant invention provides a method for obtaining ssDNA directly from dsPCR products. The method is significantly faster than previously available techniques, and is able to simultaneously generate highly purified ssDNA of a specific size from the PCR products. The invention thus represents a substantial advance in the field of molecular biology.

### SUMMARY OF THE INVENTION

The instant invention provides a novel method for generating single stranded DNA (ssDNA) directly from double stranded PCR (dsPCR) products. The method of the invention generally entails the following steps: (1) amplifying a target polynucleotide by means of two oligonucleotide primers, wherein one primer is capable of hybridizing to the target polynucleotide and the other primer is capable of hybridizing to the complement of the target polynucleotide, and wherein one of the primers comprises a chemical tag, thereby producing an amplification product mixture comprising a tagged amplification product of the target polynucleotide and a complementary non-tagged amplification product; (2) applying the amplification product mixture to a separation medium, wherein the chemical tag is capable of interacting with the separation medium; and (3) eluting the amplification products from the separation medium by means of a mobile phase under denaturing conditions, wherein the interaction between the tag and the separation medium results in the physical separation of the tagged amplification product from the non-tagged amplification product.

In preferred embodiments of the invention, at least one of the amplification products is detected and/or collected.

In a preferred embodiment of the invention, the amplification product mixture is applied in the presence of a first counterion agent, the separation medium has a nonpolar surface, and the mobile phase contains an organic solvent.

In another preferred embodiment of the invention, the separation medium has a nonpolar separation surface that is substantially free of multivalent cations that are capable of interfering with polynucleotide separations.

In another preferred embodiment of the invention, the solutions used are substantially free of multivalent cations capable of interfering with polynucleotide separations.

It is preferably that the chemical tag has an affinity towards the separation medium.

In preferred embodiments of the invention, the chemical tag is hydrophobic. Suitable tags can comprise a hydrocarbon group, such as alkyl, cycloalkyl, aryl or arylalkyl groups. In particularly preferred embodiments the chemical tag is a fluorescent group. An especially preferred tag is biotin.

In other embodiments of the invention the chemical tag is not hydrophobic, and, for example, can be a charged group.

In another preferred embodiment of the invention, the separation medium comprises polymer beads having an average diameter of 0.5 to 100 microns, the beads being unsubstituted polymer beads or polymer beads substituted with a moiety selected from the group consisting of hydrocarbon having from one to 1,000,000 carbons.

In still another preferred embodiment of the invention, the separation medium comprises particles selected from the group consisting of silica, silica carbide, silica nitrite, titanium oxide, aluminum oxide, zirconium oxide, carbon, insoluble polysaccharide, and diatomaceous earth, the particles having separation surfaces which are coated with a hydrocarbon or non-polar hydrocarbon substituted polymer, or have substantially all polar groups reacted with a non-polar hydrocarbon or substituted hydrocarbon group, wherein the surfaces are non-polar.

In yet another preferred embodiment of the invention, the medium comprises a monolith, such as a polymeric monolith or a derivatized silica gel monolith.

Suitable tags can comprise a hydrocarbon group, such as alkyl, cycloalkyl, aryl or arylalkyl groups the separation medium has been subjected to acid wash treatment to remove any residual surface metal contaminants.

In still yet another preferred embodiment of the invention, the separation medium has been subjected to treatment with a multivalent cation binding agent.

In still another preferred embodiment of the invention, the organic solvent is selected from the group consisting of alcohol, nitrile, dimethylformamide, tetrahydrofuran, ester, ether, and mixtures of one or more thereof. Acetonitrile is a particularly preferred organic solvent

In yet another preferred embodiment of the invention, the mobile phase contains a second counterion agent, which may or may not be the same as the first counterion agent.

In still another preferred embodiment of the invention, the first and second counterion agents are selected from the group consisting of lower alkyl primary amine, lower alkyl secondary amine, lower alkyl tertiary amine, lower trialkylammonium salt, quaternary ammonium salt, and mixtures of one or more thereof.

Preferred counterion agents are selected from the group consisting of octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyldiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, tetrabutylammonium acetate, triethylammonium hexafluoroisopropyl alcohol, and mixtures of one or more thereof. Particularly preferred counterion agents include tetrabutylammonium acetate and triethylammonium acetate.

A preferred means of DNA amplification is PCR. Typically the amplification products are DNA molecules.

### BRIEF DESCRIPTION OF THE DRAWINGS

FIG. 1 is a chromatogram showing the separation of biotinylated and non-biotinylated oligodeoxynucleotides of the same length by IP-RP-DHPLC.

FIG. 2 is a chromatogram showing the separation of a 200 bp asymmetrically biotinylated DNA duplex

FIG. 3 show the chromatographic separation of an asymmetrically biotinylated random DNA pool. Under non-denaturing conditions (45° C.) the amplified ds DNA is eluted as a single broad peak (FIG. 3a), while under denaturing conditions (75° C.) the two different ssDNA species are separated owing to the influence of the biotin moiety (FIG. 3b).

FIG. 4 shows a IP-RP-DHPLC analysis of an asymemetrically labeled fluorescent duplex (6-FAM) of 50 nucleotides in length.

FIG. 4a shows the UV analysis and

FIG. 4b the fluorescent analysis. The chromatogram shows the presence of the fluorescent ssDNA at the longer retention time.

FIG. 5 is a chromatogram showing the separation of a 200 bp asymmetrically fluorescently labeled PCR product.

FIG. 5a shows the UV analysis and

FIG. 5b the fluorescent analysis. The chromatogram shows the presence of the fluorescnet ssDNA at the longer retention time; the peaks seen before the main fluorescent ssDNA are primer dimer PCr artefacts.

### DETAILED DESCRIPTION OF THE INVENTION

The instant invention provides a novel method for generating single stranded DNA (ssDNA) directly from double stranded PCR (dsPCR) products. The method of the invention generally entails the following steps: (1) amplifying a target polynucleotide by means of two oligonucleotide primers, wherein one primer is capable of hybridizing to the target polynucleotide and the other primer is capable of hybridizing to the complement of the target polynucleotide, and wherein one of the primers comprises a chemical tag, thereby producing an amplification product mixture comprising a tagged amplification product of the target polynucleotide and a complementary non-tagged amplification product; (2) applying the amplification product mixture to a separation medium, wherein the chemical tag is capable of interacting with the separation medium; and (3) eluting the amplification products from the separation medium by means of a mobile phase under denaturing conditions, wherein the interaction between the tag and the separation medium results in the physical separation of the tagged amplification product from the non-tagged amplification product. The method is described in more detail below.

As used herein, the term “polynucleotide” is defined as a polymer containing an indefinite number of nucleotides, linked from one ribose (or deoxyribose) to another via phosphodiester bonds. The polynucleotide can be a linear molecule or a closed circle and can be modified, e.g. labeled with biotin or fluorescent molecules. A polynucleotide can be single-stranded or double-stranded. Short, typically single-stranded polynucleotides are referred to as oligonucleotides, and are often used in molecular biology as primers and probes. DNA and RNA, both naturally occurring as well as synthetic derivatives and analogs (e.g., DNA analogs containing modified nucleotides), are particularly important examples of polynucleotides of relevance to the instant invention.

The term “products of an amplification reaction” refers to the polynucleotide products of a molecular biology reaction capable of generating multiple copies of a template polynucleotide. The preferred mode of amplification in the practice of the present invention is polymerase chain reaction (PCR), as described in U.S. Pat. Nos. 4,683,194, 4,683,195 and 4,683,202, which are incorporated herein by reference. This process involves the use of pairs of primers, one for each strand of the duplex DNA, that will hybridize at a site located near a region of interest in a poynucleotide. Chain extension polymerization is then carried out in repetitive cycles to increase the number of copies of the region of interest many times. Gelfand et al. have described a thermostable enzyme, “Taq polymerase,” derived from the organism Thermus aquaticus, which is useful in this amplification process. (See U.S. Pat. Nos. 4,889,818; 5,352,600 and 5,079,352 which are incorporated herein by reference). In general, the product of a PCR is dsDNA, comprising two complementary polynucleotide strands. The two strands are hybridized to one another under native, non-denaturing conditions, but under denaturing conditions the strands can dissociate to form two ssDNA molecules.

As used herein, the term “target polynucleotide” refers to a segment of polynucleotide that is amplified, either selectively using a sequence specific primer (or primers), or randomly using random primers. A “primer” is an oligonucleotide capable of hybridizing to a complementary stretch of polynucleoitde and initiating replication by an enzyme with DNA polymerase activity (e.g., DNA polymerase, reverse transcriptase).

Although PCR is the preferred amplification method, amplification of target sequences in a sample may be accomplished by any known method, such as ligase chain reaction (Wu and Wallace 1988, Genomics 4:560-569, incorporated herein by reference), the TAS amplification system (Kwoh et al., 1989, Proc. Natl. Acad. Sci. USA 86:1173-1177, incorporated herein by reference), and self-sustained sequence replication (Guatelli et al., 1990, Proc. Natl. Acad. Sci. USA 87:1874-1878, incorporated herein by reference), each of which provides sufficient amplification so that the target sequence can be detected by nucleic acid hybridization to an SSO probe. Alternatively, methods that amplify the probe to detectable levels can be used, such as Q.beta.-replicase amplification (Kramer and Lizardi, 1989, Nature 339:401-402, and Lorneli et al., 1989, Clin. Chem. 35:1826-1831, both of which are incorporated herein by reference). A review of known amplification methods is provided in Abramson and Myers, 1993, Current Opinion in Biotechnology 4:41-47, incorporated herein by reference. The term “probe,” as used herein, encompasses the sequence-specific oligonucleotides used in the above procedures; for instance, the two or more oligonucleotides used in LCR are “probes” for purposes of the present invention, even though some embodiments of LCR only require ligation of the probes to indicate the presence of an allele.

The term “chemical tag” refers to a chemical moiety attached to a polynucleotide, such as a primer or an amplification product. Non-limiting examples of tags include hydrophobic moieties, ionic moieties, dyes, etc. In a preferred embodiment of the invention, the biotin serves as a chemical tag. For use in the present invention, a chemical tag should be capable of interacting with the separation medium, either directly or indirectly (e.g., through an intermediary). In preferred embodiments of the invention, the chemical tag has an affinity for the separation medium, i.e., some chemical attraction or tendency to bind to the separation medium.

The term “counterion agent” is defined herein as a compound used to form a ionic pair with a polynucleotide that is capable of separation by the methods described herein. Preferred counterion agents comprise a cationic species having a hydrophobic character (e.g., an alkylated cation such as triethylammonium), believed to be capable of forming a bridging interaction between negatively charged polynucleotides and the hydrophobic surface of a separation medium of the invention.

The term “denaturing conditions” refers to conditions where double stranded amplification products are denatured, i.e., the strands lose their ability to hybridize to their complement, and are able to dissociate into single stranded polynucleotides. Denaturing conditions can be achieved, for example, by conducting chromatography at high temperature (usually at about 50° C. or greater, preferably at about 50° C. or greater, and most preferably at about 75° C. or greater), at a pH sufficient to cause denaturation, in the presence of a chemical denaturant, or a combination thereof. Elevated temperature in the preferred means of achieving denaturing conditions.

“Separation medium” refers to a solid phase having a hydrophobic surface suitable for binding polynucleotides in the presence of an aqueous phase containing a suitable counterion agent. Examples include beads, particles and monoliths.

The term “organic solvent” refers to a solvent of sufficient non-polar character to cause elution of a polynucleotide from a separation medium when used as a component of an mobile phase. Preparation of a mobile phase is facilitated by the used of an organic solvent that is suitably water-soluble.

The term “physical separation” as applied to amplification products refers to separation of the two complementary strands of the amplification product such that the individual strands can be physically isolated from one another.

Ion-Pairing Reversed-Phase Chromatography (IP-RPC) is a powerful form of chromatography used in the separation and analysis of polynucleotides, including DNA (both single and double stranded) and RNA (Eriksson et al., (1986) J. Chromatography 359:265-74). Most reported applications of IP-RPC have been in the context of high performance liquid chromatography (IP-RP-HPLC), but the technology can be accomplished using non-HPLC chromatography systems (U.S. patent application Ser. Nos. 09/318,407 and 09/391,963). Nevertheless, for the sake of simplicity the following description will focus primarily on the use of IP-RP-HPLC, a particularly powerful and convenient form of IP-RPC. It is to be understood that this is not intended to limit the scope of the invention, and that generally the methods described can be performed without the use of HPLC, although this will in some cases lead to less than optimal results. IP-RPC is a form of chromatography characterized by the use of a reversed phase (i.e., hydrophobic or non-polar, the two terms being used interchangeably herein) stationary phase and a mobile phase that includes an alkylated cation (e.g., triethylammonium) that is believed to form a bridging interaction between the negatively charged polynucleotide and non-polar stationary phase. The alkylated cation-mediated interaction of polynucleotide and stationary phase can be modulated by the polarity of the mobile phase, conveniently adjusted by means of a solvent that is less polar than water, e.g., acetonitrile. In general, a polynucleotide such as RNA is retained by the separation medium in the presence of counterion agent, and can be eluted by increasing the concentration of a non-polar solvent, Elution can be accomplished in the presence or absence of counterion agent. Performance is enhanced by the use of a non-porous separation medium, as described in U.S. Pat. No. 5,585,236. A superior form of IP-RP-HPLC, termed Matched Ion Polynucleotide Chromatography (MIPC), is described in U.S. Pat. Nos. 5,585,236, 6,066,258 and 6,056,877 and PCT Publication Nos. WO98/48913, WO98/48914, WO/9856797, WO98/56798, incorporated herein by reference in their entirety. MIPC is characterized by the use of solvents and chromatographic surfaces that are substantially free of multivalent cation contamination that can interfere with polynucleotide separation. In the practice of the instant invention, a preferred system for performing MIPC separations is that provided by Transgenomic, Inc. under the trademark WAVE®.

In the instant invention, separation is preferably achieved under denaturing conditions using denaturing RP-IP-HPLC (RP-IP-DHPLC), a technique described by Huber et al. (1995) Anal. Chem. 67:578-85).

Separation by RP-IP-HPLC, including MIPC, occurs at the non-polar surface of a separation medium. In one embodiment, the non-polar surfaces comprise the surfaces of polymeric beads. In an alternative embodiment, the surfaces comprise the surfaces of interstitial spaces in a molded polymeric monolith, described in more detail infra. For purposes of simplifying the description of the invention and not by way of limitation, the separation of polynucleotides using nonporous beads, and the preparation of such beads, will be primarily described herein, it being understood that other separation surfaces, such as the interstitial surfaces of polymeric monoliths, are intended to be included within the scope of this invention.

In general, in order to be suitable for use in IP-RP-HPLC a separation medium should have a surface that is either intrinsically non-polar or bonded with a material that forms a surface having sufficient non-polarity to interact with a counterion agent.

In one aspect of the invention, IP-RP-HPLC detection is accomplished using a column filled with nonporous polymeric beads having an average diameter of about 0.5-100 microns; preferably, 1-10 microns; more preferably, 1-5 microns. Beads having an average diameter of 1.0-3.0 microns are most preferred.

In a preferred embodiment of the invention, the chromatographic separation medium comprises nonporous beads, i.e., beads having a pore size that essentially excludes the polynucleotides being separated from entering the bead, although porous beads can also be used. As used herein, the term “nonporous” is defined to denote a bead that has surface pores having a diameter that is sufficiently small so as to effectively exclude the smallest DNA fragment in the separation in the solvent medium used therein. Included in this definition are polymer beads having these specified maximum size restrictions in their natural state or which have been treated to reduce their pore size to meet the maximum effective pore size required.

The surface conformations of nonporous beads of the present invention can include depressions and shallow pit-like structures that do not interfere with the separation process. A pretreatment of a porous bead to render it nonporous can be effected with any material which will fill the pores in the bead structure and which does not significantly interfere with the MIPC process.

Pores are open structures through which mobile phase and other materials can enter the bead structure. Pores are often interconnected so that fluid entering one pore can exit from another pore. Without intending to be bound by any particular theory, it is believed that pores having dimensions that allow movement of the polynucleotide into the interconnected pore structure and into the bead impair the resolution of separations or result in separations that have very long retention times.

Non-porous polymeric beads useful in the practice of the present invention can be prepared by a two-step process in which small seed beads are initially produced by emulsion polymerization of suitable polymerizable monomers. The emulsion polymerization procedure is a modification of the procedure of Goodwin, et al. (Colloid & Polymer Sci., 252:464-471 (1974)). Monomers which can be used in the emulsion polymerization process to produce the seed beads include styrene, alkyl substituted styrenes, alpha-methyl styrene, and alkyl substituted alpha-methyl styrene. The seed beads are then enlarged and, optionally, modified by substitution with various groups to produce the nonporous polymeric beads of the present invention.

The seed beads produced by emulsion polymerization can be enlarged by any known process for increasing the size of the polymer beads. For example, polymer beads can be enlarged by the activated swelling process disclosed in U.S. Pat. No. 4,563,510. The enlarged or swollen polymer beads are further swollen with a crosslinking polymerizable monomer and a polymerization initiator. Polymerization increases the crosslinking density of the enlarged polymeric bead and reduces the surface porosity of the bead. Suitable crosslinking monomers contain at least two carbon-carbon double bonds capable of polymerization in the presence of an initiator. Preferred crosslinking monomers are divinyl monomers, preferably alkyl and aryl (phenyl, naphthyl, etc.) divinyl monomers and include divinyl benzene, butadiene, etc. Activated swelling of the polymeric seed beads is useful to produce polymer beads having an average diameter ranging from 1 up to about 100 microns.

Alternatively, the polymer seed beads can be enlarged simply by heating the seed latex resulting from emulsion polymerization. This alternative eliminates the need for activated swelling of the seed beads with an activating solvent. Instead, the seed latex is mixed with the crosslinking monomer and polymerization initiator described above, together with or without a water-miscible solvent for the crosslinking monomer. Suitable solvents include acetone, tetrahydrofuran (THF), methanol, and dioxane. The resulting mixture is heated for about 1-12 hours, preferably about 4-8 hours, at a temperature below the initiation temperature of the polymerization initiator, generally, about 10° C.-80° C., preferably 30° C.-60° C. Optionally, the temperature of the mixture can be increased by 10-20% and the mixture heated for an additional 1 to 4 hours. The ratio of monomer to polymerization initiator is at least 100:1, preferably in the range of about 100:1 to about 500:1, more preferably about 200:1 in order to ensure a degree of polymerization of at least 200. Beads having this degree of polymerization are sufficiently pressure-stable to be used in HPLC applications. This thermal swelling process allows one to increase the size of the bead by about 110-160% to obtain polymer beads having an average diameter up to about 5 microns, preferably about 2-3 microns. The thermal swelling procedure can, therefore, be used to produce smaller particle sizes previously accessible only by the activated swelling procedure.

Following thermal enlargement, excess crosslinking monomer is removed and the particles are polymerized by exposure to ultraviolet light or heat. Polymerization can be conducted, for example, by heating of the enlarged particles to the activation temperature of the polymerization initiator and continuing polymerization until the desired degree of polymerization has been achieved. Continued heating and polymerization allows one to obtain beads having a degree of polymerization greater than 500.

For use in the present invention, packing material disclosed by U.S. Pat. No. 4,563,510 can be modified through substitution of the polymeric beads with alkyl groups or can be used in its unmodified state. For example, the polymer beads can be alkylated with 1 or 2 carbon atoms by contacting the beads with an alkylating agent, such as methyl iodide or ethyl iodide. Alkylation can be achieved by mixing the polymer beads with the alkyl halide in the presence of a Friedel-Crafts catalyst to effect electrophilic aromatic substitution on the aromatic rings at the surface of the polymer blend. Suitable Friedel-Crafts catalysts are well-known in the art and include Lewis acids such as aluminum chloride, boron trifluoride, tin tetrachloride, etc. The beads can be hydrocarbon substituted by substituting the corresponding hydrocarbon halide for methyl iodide in the above procedure, for example.

The term alkyl as used herein in reference to the beads useful in the practice of the present invention is defined to include alkyl and alkyl substituted aryl groups, having from 1 to 1,000,000 carbons, the alkyl groups including straight chained, branch chained, cyclic, saturated, unsaturated nonionic functional groups of various types including aldehyde, ketone, ester, ether, alkyl groups, and the like, and the aryl groups including as monocyclic, bicyclic, and tricyclic aromatic hydrocarbon groups including phenyl, naphthyl, and the like. Methods for alkyl substitution are conventional and well-known in the art and are not an aspect of this invention. The substitution can also contain hydroxy, cyano, nitro groups, or the like which are considered to be non-polar, reverse phase functional groups.

Non-limiting examples of base polymers suitable for use in producing such polymer beads include mono- and di-vinyl substituted aromatics such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(styrene-divinylbenzene) and poly(ethylvinylbenzene-divinylbenzene). Methods for making beads from these polymers are conventional and well known in the art (for example, see U.S. Pat. No. 4,906,378). The physical properties of the surface and near-surface areas of the beads are the primary determinant of chromatographic efficiency. The polymer, whether derivatized or not, should provide a nonporous, non-reactive, and non-polar surface for the MIPC separation. In a particularly preferred embodiment of the invention, the separation medium consists of octadecyl modified, nonporous alkylated poly(styrene-divinylbenzene) beads. Separation columns employing these particularly preferred beads, referred to as DNASep® columns, are commercially available from Transgenomic, Inc.

A separation bead used in the invention can comprise a nonporous particle which has non-polar molecules or a non-polar polymer attached to or coated on its surface. In general, such beads comprise nonporous particles which have been coated with a polymer or which have substantially all surface substrate groups reacted with a non-polar hydrocarbon or substituted hydrocarbon group, and any remaining surface substrate groups endcapped with a tri(lower alkyl)chlorosilane or tetra(lower alkyl)dichlorodisilazane as described in U.S. Pat. No. 6,056,877.

The nonporous particle is preferably an inorganic particle, but can be a nonporous organic particle. The nonporous particle can be, for example, silica, silica carbide, silica nitrite, titanium oxide, aluminum oxide, zirconium oxide, carbon, insoluble polysaccharides such as cellulose, or diatomaceous earth, or any of these materials which have been modified to be nonporous. Examples of carbon particles include diamond and graphite which have been treated to remove any interfering contaminants. The preferred particles are essentially non-deformable and can withstand high pressures. The nonporous particle is prepared by known procedures. The preferred particle size is about 0.5-100 microns; preferably, 1-10 microns; more preferably, 1-5 microns. Beads having an average diameter of 1.0-3.0 microns are most preferred.

Because the chemistry of preparing conventional silica-based reverse phase HPLC materials is well-known, most of the description of non-porous beads suitable for use in the instant invention is presented in reference to silica. It is to be understood, however, that other nonporous particles, such as those listed above, can be modified in the same manner and substituted for silica. For a description of the general chemistry of silica, see Poole, Colin F. and Salwa K. Poole, Chromatography Today, Elsevier:N.Y. (1991), pp. 313-342 and Snyder, R. L. and J. J. Kirkland, Introduction to Modem Liquid Chromatography, 2^{nd} ed., John Wiley & Sons, Inc.:New York (1979), pp. 272-278, the disclosures of which are hereby incorporated herein by reference in their entireties.

The nonporous beads of the invention are characterized by having minimum exposed silanol groups after reaction with the coating or silating reagents. Minimum silanol groups are needed to reduce the interaction of the DNA with the substrate and also to improve the stability of the material in a high pH and aqueous environment. Silanol groups can be harmful because they can repel the negative charge of the DNA molecule, preventing or limiting the interaction of the DNA with the stationary phase of the column. Another possible mechanism of interaction is that the silanol can act as ion exchange sites, taking up metals such as iron (III) or chromium (III). Iron (III) or other metals which are trapped on the column can distort the DNA peaks or even prevent DNA from being eluted from the column.

Silanol groups can be hydrolyzed by the aqueous-based mobile phase. Hydrolysis will increase the polarity and reactivity of the stationary phase by exposing more silanol sites, or by exposing metals that can be present in the silica core. Hydrolysis will be more prevalent with increased underivatized silanol groups. The effect of silanol groups on the DNA separation depends on which mechanism of interference is most prevalent. For example, iron (III) can become attached to the exposed silanol sites, depending on whether the iron (III) is present in the eluent, instrument or sample.

The effect of metals can only occur if metals are already present within the system or reagents. Metals present within the system or reagents can get trapped by ion exchange sites on the silica. However, if no metals are present within the system or reagents, then the silanol groups themselves can cause interference with DNA separations. Hydrolysis of the exposed silanol sites by the aqueous environment can expose metals that might be present in the silica core.

Fully hydrolyzed silica contains a concentration of about 8 μmoles of silanol groups per square meter of surface. At best, because of steric considerations, a maximum of about 4.5 μmoles of silanol groups per square meter can be reacted, the remainder of the silanol being sterically shielded by the reacted groups. Minimum silanol groups is defined as reaching the theoretical limit of or having sufficient shield to prevent silanol groups from interfering with the separation.

Numerous methods exist for forming nonporous silica core particles. For example, sodium silicate solution poured into methanol will produce a suspension of finely divided spherical particles of sodium silicate. These particles are neutralized by reaction with acid. In this way, globular particles of silica gel are obtained having a diameter of about 1-2 microns. Silica can be precipitated from organic liquids or from a vapor. At high temperature (about 2000° C.), silica is vaporized, and the vapors can be condensed to form finely divided silica either by a reduction in temperature or by using an oxidizing gas. The synthesis and properties of silica are described by R. K. Iler in The Chemistry of Silica, Solubility, Polymerization, Colloid and Surface Properties, and Biochemistry, John Wiley & Sons:New York (1979).

W. Stöber et al. described controlled growth of monodisperse silica spheres in the micron size range in J. Colloid and Interface Sci., 26:62-69 (1968). Stöber et al. describe a system of chemical reactions which permit the controlled growth of spherical silica particles of uniform size by means of hydrolysis of alkyl silicates and subsequent condensation of silicic acid in alcoholic solutions. Ammonia is used as a morphological catalyst. Particle sizes obtained in suspension range from less than 0.05 μm to 2 μm in diameter.

To prepare a nonporous bead, the nonporous particle can be coated with a polymer or reacted and endcapped so that substantially all surface substrate groups of the nonporous particle are blocked with a non-polar hydrocarbon or substituted hydrocarbon group. This can be accomplished by any of several methods described in U.S. Pat. No. 6,056,877. Care should be taken during the preparation of the beads to ensure that the surface of the beads has minimum silanol or metal oxide exposure and that the surface remains nonporous. Nonporous silica core beads can be obtained from Micra Scientific (Northbrook, Ill.) and from Chemie Uetikkon (Lausanne, Switzerland).

In another embodiment of the present invention, the IP-RP-HPLC separation medium can be in the form of a polymeric monolith, e.g., a rod-like monolithic column. A monolith is a polymer separation media, formed inside a column, having a unitary structure with through pores or interstitial spaces that allow eluting solvent and analyte to pass through and which provide the non-polar separation surface, as described in U.S. Pat. No. 6,066,258 and U.S. patent application Ser. No. 09/562,069. The interstitial separation surfaces can be porous, but are preferably nonporous. The separation principles involved parallel those encountered with bead-packed columns. As with beads, pores traversing the monolith must be compatible with and permeable to DNA.

In a preferred embodiment, the rod is substantially free of contamination capable of reacting with DNA and interfering with its separation, e.g., multivalent cations.

A molded polymeric monolith rod that can be used in practicing the present invention can be prepared, for example, by bulk free radical polymerization within the confines of a chromatographic column. The base polymer of the rod can be produced from a variety of polymerizable monomers. For example, the monolithic rod can be made from polymers, including mono- and di-vinyl substituted aromatic compounds such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(glycidyl methacrylate-co-ethylene dimethacrylate), poly(styrene-divinylbenzene) and poly(ethylvinylbenzene-divinylbenzene. The rod can be unsubsituted or substituted with a substituent such as a hydrocarbon alkyl or an aryl group. The alkyl group optionally has 1 to 1,000,000 carbons inclusive in a straight or branched chain, and includes straight chained, branch chained, cyclic, saturated, unsaturated nonionic functional groups of various types including aldehyde, ketone, ester, ether, alkyl groups, and the like, and the aryl groups includes as monocyclic, bicyclic, and tricyclic aromatic hydrocarbon groups including phenyl, naphthyl, and the like. In a preferred embodiment, the alkyl group has 1-24 carbons. In a more preferred embodiment, the alkyl group has 1-8 carbons. The substitution can also contain hydroxy, cyano, nitro groups, or the like which are considered to be non-polar, reverse phase functional groups. Methods for hydrocarbon substitution are conventional and well-known in the art and are not an aspect of this invention. The preparation of polymeric monoliths is by conventional methods well known in the art as described in the following references: Wang et al.(1994) J. Chromatog. A 699:230; Petro et al. (1996) Anal. Chem. 68:315 and U.S. Pat. Nos. 5,334,310; 5,453,185 and 5,522,994. Monolith or rod columns are commercially available form Merck & Co (Darmstadt, Germany).

The separation medium can take the form of a continuous monolithic silica gel. A molded monolith can be prepared by polymerization within the confines of a chromatographic column (e.g., to form a rod) or other containment system. A monolith is preferably obtained by the hydrolysis and polycondensation of alkoxysilanes. A preferred monolith is derivatized in order to produce non-polar interstitial surfaces. Chemical modification of silica monoliths with ocatdecyl, methyl or other ligands can be carried out. An example of a preferred derivatized monolith is one which is polyfunctionally derivatized with octadecylsilyl groups. The preparation of derivatized silica monoliths can be accomplished using conventional methods well known in the art as described in the following references which are hereby incorporated in their entirety herein: U.S. Pat. No. 6,056,877, Nakanishi, et al., J. Sol-Gel Sci. Technol. 8:547 (1997); Nakanishi, et al., Bull, Chem. Soc. Jpn. 67:1327 (1994); Cabrera, et al., Trends Analytical Chem. 17:50 (1998); Jinno, et al., Chromatographia 27:288 (1989).

MIPC is characterized by the use of a separation medium having low amounts of metal contaminants or other contaminants that can bind DNA. Preferred beads and monoliths have been produced under conditions where precautions have been taken to substantially eliminate any multivalent cation contaminants (e.g. Fe(III), Cr(III), or colloidal metal contaminants), including a decontamination treatment, e.g., an acid wash treatment. Only very pure, non-metal containing materials should be used in the production of the beads in order to minimize the metal content of the resulting beads.

In addition to the separation medium being substantially metal-free, to achieve optimum peak separation the separation column and all process solutions held within the column or flowing through the column are preferably substantially free of multivalent cation contaminants (e.g. Fe(III), Cr(III), and colloidal metal contaminants). As described in U.S. Pat. Nos. 5,772,889, 5,997,742 and 6,017,457, this can be achieved by supplying and feeding solutions that enter the separation column with components that have process solution-contacting surfaces made of material which does not release multivalent cations into the process solutions held within or flowing through the column, in order to protect the column from multivalent cation contamination. The process solution-contacting surfaces of the system components are preferably material selected from the group consisting of titanium, coated stainless steel, passivated stainless steel, and organic polymer. Metals found in stainless steel, for example, do not harm the separation, unless they are in an oxidized or colloidal partially oxidized state. For example, 316 stainless steel frits are acceptable in column hardware, but surface oxidized stainless steel frits harm the DNA separation.

For additional protection, multivalent cations in mobile phase solutions and sample solutions entering the column can be removed by contacting these solutions with multivalent cation capture resin before the solutions enter the column to protect the separation medium from multivalent cation contamination. The multivalent capture resin is preferably cation exchange resin and/or chelating resin.

Trace levels of multivalent cations anywhere in the solvent flow path can cause a significant deterioration in the resolution of the separation after multiple uses of an IP-RP-HPLC column. This can result in increased cost caused by the need to purchase replacement columns and increased downtime. Therefore, effective measures are preferably taken to prevent multivalent metal cation contamination of the separation system components, including separation media and mobile phase contacting. These measures include, but are not limited to, washing protocols to remove traces of multivalent cations from the separation media and installation of guard cartridges containing cation capture resins, in line between the mobile phase reservoir and the MIPC column. These, and similar measures, taken to prevent system contamination with multivalent cations have resulted in extended column life and reduced analysis downtime.

There are two places where multivalent-cation-binding agents, e.g., chelators, are used in MIPC separations. In one embodiment, these binding agents can be incorporated into a solid through which the mobile phase passes. Contaminants are trapped before they reach places within the system that can harm the separation. In these cases, the functional group is attached to a solid matrix or resin (e.g., a flow-through cartridge, usually an organic polymer, but sometimes silica or other material). The capacity of the matrix is preferably about 2 mequiv./g. An example of a suitable chelating resin is available under the trademark CHELEX 100 (Dow Chemical Co.) containing an iminodiacetate functional group.

In another embodiment, the multivalent cation-binding agent can be added to the mobile phase. The binding functional group is incorporated into an organic chemical structure. The preferred multivalent cation-binding agent fulfills three requirements. First, it is soluble in the mobile phase. Second, the complex with the metal is soluble in the mobile phase. Multivalent cation-binding agents such as EDTA fulfill this requirement because both the chelator and the multivalent cation-binding agent-metal complex contain charges, which makes them both water-soluble. Also, neither precipitate when acetonitrile, for example, is added. The solubility in aqueous mobile phase can be enhanced by attaching covalently bound ionic functionality, such as, sulfate, carboxylate, or hydroxy. A preferred multivalent cation-binding agent can be easily removed from the column by washing with water, organic solvent or mobile phase. Third, the binding agent must not interfere with the chromatographic process.

The multivalent cation-binding agent can be a coordination compound. Examples of preferred coordination compounds include water soluble chelating agents and crown ethers. Non-limiting examples of multivalent cation-binding agents which can be used in the present invention include acetylacetone, alizarin, aluminon, chloranilic acid, kojic acid, morin, rhodizonic acid, thionalide, thiourea, α-furildioxime, nioxime, salicylaldoxime, dimethylglyoxime, α-furildioxime, cupferron, α-nitroso-β-naphthol, nitroso-R-salt, diphenylthiocarbazone, diphenylcarbazone, eriochrome black T, PAN, SPADNS, glyoxal-bis(2-hydroxyanil), murexide, α-benzoinoxime, mandelic acid, anthranilic acid, ethylenediamine, glycine, triaminotriethylamine, thionalide, triethylenetetramine, EDTA, metalphthalein, arsonic acids, α,α′-bipyridine, 4-hydroxybenzothiazole, 8-hydroxyquinaldine, 8-hydroxyquinoline, 1,10-phenanthroline, picolinic acid, quinaldic acid, α,α′,α″-terpyridyl, 9-methyl-2,3,7-trihydroxy-6-fluorone, pyrocatechol, salicylic acid, tiron, 4-chloro-1,2-dimercaptobenzene, dithiol, mercaptobenzothiazole, rubeanic acid, oxalic acid, sodium diethyldithiocarbarbamate, and zinc dibenzyldithiocarbamate. These and other examples are described by Perrin in Organic Complexing Reagents: Structure, Behavior, and Application to Inorganic Analysis, Robert E. Krieger Publishing Co. (1964). In the present invention, a preferred multivalent cation-binding agent is EDTA.

To achieve high-resolution chromatographic separations of polynucleotides, it is generally necessary to tightly pack the chromatographic column with the solid phase polymer beads. Any known method of packing the column with a column packing material can be used in the present invention to obtain adequate high-resolution separations. Typically, a slurry of the polymer beads is prepared using a solvent having a density equal to or less than the density of the polymer beads. The column is then filled with the polymer bead slurry and vibrated or agitated to improve the packing density of the polymer beads in the column. Mechanical vibration or sonication is typically used to improve packing density.

For example, to pack a 50×4.6 mm I.D. column, 2.0 grams of beads can be suspended in 10 mL of methanol with the aid of sonication. The suspension is then packed into the column using 50 mL of methanol at 8,000 psi of pressure. This improves the density of the packed bed.

There are several types of counterions suitable for use with IP-RP-HPLC. These include a mono-, di-, or trialkylamine that can be protonated to form a positive counter charge or a quaternary alkyl substituted amine that already contains a positive counter charge. The alkyl substitutions may be uniform (for example, triethylammonium acetate or tetrapropylammonium acetate) or mixed (for example, propyidiethylammonium acetate). The size of the alkyl group may be small (methyl) or large (up to 30 carbons) especially if only one of the substituted alkyl groups is large and the others are small. For example octyldimethylammonium acetate is a suitable counterion agent. Preferred counterion agents are those containing alkyl groups from the ethyl, propyl or butyl size range.

Without intending to be bound by any particular theory, it is believed the alkyl group functions by imparting a nonpolar character to the DNA through an ion pairing process so that the DNA can interact with the nonpolar surface of the separation media. The requirements for the degree of nonpolarity of the counterion-DNA pair depends on the polarity of the separation media, the solvent conditions required for separation, the particular size and type of fragment being separated. For example, if the polarity of the separation media is increased, then the polarity of the counterion agent may have to be adjusted to match the polarity of the surface and increase interaction of the counterion-DNA pair. In general, as the size and hydrophobicity of the alkyl group is increased, the separation is less influenced by DNA sequence and base composition, but rather is based predominately on DNA sequence length.

In some cases, it may be desired to increase the range of concentration of organic solvent used to perform the separation. For example, increasing the alkyl chain length on the counterion agent will increase the nonpolarity of the counterion-DNA pair resulting in the need to either increase the concentration of the mobile phase organic solvent, or increase the strength of the organic solvent type, e.g., acetonitrile is about two times more effective than methanol for eluting DNA. There is a positive correlation between concentration of the organic solvent required to elute a fragment from the column and the length of the fragment. However, at high organic solvent concentrations, the polynucleotide can precipitate. To avoid precipitation, a more nonpolar organic solvent and/or a smaller counterion alkyl group can be used. The alkyl group on the counterion agent can also be substituted with halides, nitro groups, or the like to modulate polarity.

The mobile phase preferably contains a counterion agent. Typical counterion agents include trialkylammonium salts of organic or inorganic acids, such as lower alkyl primary, secondary, and lower tertiary amines, lower trialkyammonium salts and lower quaternary alkyalmmonium salts. Lower alkyl refers to an alkyl radical of one to six carbon atoms, as exemplified by methyl, ethyl, n-butyl, i-butyl, t-butyl, isoamyl, n-pentyl, and isopentyl. Examples of counterion agents include octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyidiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, and tetrabutylammonium acetate. Although the anion in the above examples is acetate, other anions may also be used, including carbonate, phosphate, sulfate, nitrate, propionate, formate, chloride, and bromide, or any combination of cation and anion. These and other agents are described by Gjerde, et al. in Ion Chromatography, 2nd Ed., Dr. Alfred Hüthig Verlag Heidelberg (1987). In a particularly preferred embodiment of the invention the counterion is tetrabutylammonium bromide (TBAB) is preferred, although other quaternary ammonium reagents such as tetrapropyl or tetrabutyl ammonium salts can be used. Alternatively, a trialkylammonium salt, e.g., triethylammonium acetate (TEM) can be used.

The pH of the mobile phase is preferably within the range of about pH 5 to about pH 9, and optimally within the range of about pH 6 to about pH 7.5.

To achieve optimum peak resolution during the separation of DNA by IP-RPHPLC, the method is preferably performed at a temperature within the range of 20° C. to 90° C.; more preferably, 30° C. to 80° C.; most preferably, 50° C. to 75° C. In practice of the instant invention, it is important to use a temperature sufficient to achieve denaturing conditions. The flow rate is selected to yield a back pressure not exceeding 5000 psi. In general, separation of single-stranded fragments should be performed at higher temperatures. In a preferred embodiment of the invention, the separation is achieved at a temperature at which the amplified extension product is denatured. The temperature required to achieve denaturation will vary, depending upon the nature of the column, the mobile phase and counterion agent used, and the melting properties of the DNA being separated. In a particularly preferred embodiment of the invention, where the separation medium is octadecyl modified, nonporous alkylated poly(styrene-divinylbenzene) beads, the aqueous mobile phase contains acetonitrile and TBAB is used as a counterion, the column temperature is preferably greater than 50° C., more preferably between about 50° C. and 80° C., and most preferably about 75° C.

The temperature at which the separation is performed affects the choice of organic solvents used in the separation, and vice versa. The solvent affects the temperature at which a double stranded DNA will melt to form two single strands or a partially melted complex of single and double stranded DNA, i.e., some solvents will stabilize a DNA duplex better than others. Furthermore, the polarity of a solvent affects the distribution of the DNA between the mobile phase and the stationary phase.

An organic solvent that is water soluble is preferably used, e.g., an alcohol, nitrile, dimethylformamide (DMF), tetrahydrofuran (THF), ester, or ether. Water soluble solvents are defined as those that exist as a single phase with aqueous systems under all conditions of operation of the present invention. For example, acetonitrile and 1-propanol have polarity and solubility properties that are particularly suited for use in the present invention. However, methanol can be a good alternative that reduces cost and toxicity concerns. Solvents that are particularly preferred for use in the method of this invention include methanol, ethanol, 2-propanol, 1-propanol, tetrahydrofuran (THF), and acetonitrile, with acetonitrile being most preferred overall.

In performing IP-RP-HPLC and MIPC, even trace levels of multivalent cations anywhere in the solvent flow path can cause a significant deterioration in the resolution of the separation after multiple uses of a column. This can result in increased cost caused by the need to purchase replacement columns and increased downtime. Therefore, effective measures are preferably taken to prevent multivalent metal cation contamination of the separation system components, including separation media and mobile phase contacting. These measures include, but are not limited to, washing protocols to remove traces of multivalent cations from the separation media and installation of guard cartridges containing cation capture resins, in line between the mobile phase reservoir and the column. These, and similar measures, taken to prevent system contamination with multivalent cations have resulted in extended column life and reduced analysis downtime.

In some instances, in order to optimize column life and maintain effective separation performance, it will be desirable to periodically run an aqueous solution of multivalent cation-binding agent through the column, e.g., after about 500 uses or when the performance starts to degrade. Examples of suitable cation-binding agents are as described hereinabove.

The concentration of a solution of the cation-binding agent can be between 0.01M and 1M. In a preferred embodiment, the column washing solution contains EDTA at a concentration of about 0.03 to 0.1M.

In another embodiment, the solution contains an organic solvent selected from the group consisting of acetonitrile, ethanol, methanol, 2-propanol, and ethyl acetate. A preferred solution contains at least 2% organic solvent to prevent microbial growth. In a most preferred embodiment a solution containing 25% acetonitrile is used to wash a column. The multivalent cation-binding solution can contain a counterion agent as described hereinabove.

In one embodiment of a column washing procedure, the separation column is washed with the multivalent cation-binding solution at an elevated temperature in the range of 50° C. to 80° C. In a preferred embodiment the column is washed with a solution containing EDTA, TEAA, and acetonitrile, in the 70° C. to 80° C. temperature range. In a specific embodiment, the solution contains 0.032 M EDTA, 0.1 M TEAA, and 25% acetonitrile.

Column washing can range from 30 seconds to one hour. In a preferred procedure, the column is washed with multivalent cation-binding agent for 30 to 60 minutes at a flow rate preferably in the range of about 0.05 to 1.0 mL/min.

Other treatments for washing a column can also be used alone or in combination with those indicated hereinabove. These include: use of high pH washing solutions (e.g., pH 10-12), use of denaturants such as urea or formamide, and reverse flushing the column with washing solution.

MIPC separation efficiency can be preserved by storing the column separation media in the presence of a solution of multivalent cation-binding agent. The solution of binding agent may also contain a counterion agent. Any of the multivalent cation-binding agents, counterion agents, and solvents described hereinabove are suitable for the purpose of storing a MIPC column. In a preferred embodiment, a column packed with MIPC separation media is stored in an organic solvent containing a multivalent cation-binding agent and a counterion agent. An example of this preferred embodiment is 0.032 M EDTA and 0.1M TEAA in 25% aqueous acetonitrile. In preparation for storage, a solution of multivalent cation-binding agent, as described above, is passed through the column for about 30 minutes. The column is then disconnected from the HPLC apparatus and the column ends are capped with commercially available threaded end caps made of material which does not release multivalent cations. Such end caps can be made of coated stainless steel, titanium, organic polymer or any combination thereof.

High pressure pumps are used for pumping mobile phase in the systems described in U.S. Pat. No. 5,585,236 to Bonn and in U.S. Pat. No. 5,772,889 to Gjerde. It will be appreciated that other methods are known for driving mobile phase through separation media and can be used in carrying out the analysis described in the present invention. A non-limiting example of such an alternative method includes “capillary electrochromatography” (CEC) in which an electric field is applied across capillary columns packed with microparticles and the resulting electroosmotic flow acts as a pump for chromatography. Electroosmosis is the flow of liquid, in contact with a solid surface, under the influence of a tangentially applied electric field. The technique combines the advantages of the high efficiency obtained with capillary electrophoretic separations, such as capillary zone electrophoresis, and the general applicability of HPLC. CEC has the capability to drive the mobile phase through columns packed with chromatographic particles, especially small particles, when using electroosmotic flow. High efficiencies can be obtained as a result of the plug-like flow profile. In the use of CEC in the present invention, solvent gradients are used and rapid separations can be obtained using high electric fields. The following references describing CEC are each incorporated in their entirety herein: Dadoo, et al, LC-GC 15:630 (1997); Jorgenson, et al., J. Chromatog. 218:209 (1981); Pretorius, et al., J. Chromatog. 99:23 (1974); and the following to Dadoo U.S. Pat. Nos. 5,378,334 (1995), 5,342,492 (1994), and 5,310,463 (1994). In the operation of this aspect of the present invention, the capillaries are packed, either electrokinetically or using a pump, with the separation beads described in the present specification. In another embodiment, a polymeric rod is prepared by bulk free radical polymerization within the confines of a capillary column. Capillaries are preferably formed from fused silica tubing or etched into a block. The packed capillary (e.g., a 150-μm i.d. with a 20-cm packed length and a window located immediately before the outlet frit) is fitted with frits at the inlet and outlet ends. An electric field, e.g., 2800V/cm, is applied. Detection can be by uv absorbance or by fluorescence. A gradient of organic solvent, e.g., acetonitrile, is applied in a mobile phase containing counterion agent (e.g. 0.1 M TEM). to elute the polynucleotides. The column temperature is maintained by conventional temperature control means. In the preferred embodiment, all of the precautions for minimizing trace metal contaminants as described hereinabove are employed in using CEC.

The chemical tag used in the present invention can be a fluorescent group, preferably a chemical which absorbs at a wavelength different from the polynucleotide itself. Non-limiting examples of fluorescent groups suitable for use with the instant invention include 5-carboxyfluorescein (5-FAM), 6-carboxyfluorescein (6-FAM), 2′,7′-dimethoxy-4′,5′-dichloro-6-carboxyfluorescein (JOE), N,N,N′-N-tetramethyl-6-carboxy rhodamine (TAMRA), 6-carboxy-X-rhodamine (ROX), 4,7,2′,4′,5′,7′-hexachloro-6-carboxy-fluorescein (HEX-1), 4,7,2′,4′,5′,7′-hexachloro-5-carboxy-fluorescein (HEX-2), 2′,4′,5′,7′-tetrachloro-5-carboxy-fluorescein (ZOE), 4,7,2′,7′-tetrachloro-6-carboxyfluorescein (TET-1), 1′,2′,7′,8′-dibenzo-4,7-dichloro-5-carboxyfluorescein (NAN-2), and 1′,2′,7′,8′-dibenzo-4,7-dichloro-6-carboxyfluorescein, fluorescein and fluorescein derivatives, Rhodamine, Cascade Blue, Alexa₃₅₀, Alexa₄₈₈, phycoerythrin, allophycocyanin, phycocyanin, rhodamine, Texas Red, EDANS, BODIPY dyes such as BODIPY-FL and BODIPY-TR-X, tetramethylrhodamine, Cy3 and Cy5, 5,6-carboxyfluorescein, fluorescein mono-derivatized with a linking functionality at either the 5 or 6 carbon position, including fluorescein-5-isothiocyanate, fluorescein-6-isothiocyanate (the -5- and -6-forms being referred to collectively as FITC), fluorescein-5-succinimidylcarboxylate, fluorescein-6-succinimidylcarboxylate, fluorescein-5-iodoacetamide, fluorescein-6-iodoacetamide, fluorescein-5-maleimide, and fluorescein-6-maleimide; 2′,7′-dimethoxy4′,5′-dichlorofluorescein mono-derivatized with a linking functionality at the 5 or 6 carbon position, including 2′,7′-dimethoxy-4′,5′-dichlorofluorescein-5-succinimidylcarboxylate and 2′,7′-dimethoxy-4′,5′-dichlorofluoescein-6-succinimidylcarboxylate (the -5- and -6-forms being referred to collectively as DDFCS), tetramethylrhodamine mono-derivatized with a linking functionality at either the 5 or 6 carbon position, including tetramethylrhodamine-5-isothiocyanate, tetramethylrhodamine-6-isothiocyanate (the -5- and -6-forms being referred to collectively as TMRITC), tetramethylrhodamine-5-iodoacetamide, tetramethylrhodamine-6-iodoacetamide, tetramethyl rhodamine-5-succinimidylcarboxylate, tetramethylrhodamine-6-succinimidylcarboxylate, tetramethylrhodamine-5-maleimide, and tetramethylrhodamine-6-maleimide, rhodamine X derivatives having a disubstituted phenyl attached to the molecule's oxygen heterocycle, one of the substituents being a linking functionality attached to the 4′ or 5′ carbon (IUPAC numbering) of the phenyl, and the other being a acidic anionic group attached to the 2′ carbon, including Texas Red (tradename of Molecular Probes, Inc.), rhodamine X-5-isothiocyanate, rhodamine X-6-isothiocyanate, rhodamine X-5-iodoacetamide, rhodamine X-6-iodoacetamide, rhodamine X-5-succinimidylcarboxylate, rhodamine X-6-succinimidylcarboxylate, rhodamine X-5-maleimide, and rhodamine X-6-maleimide (see, e.g., Brandis (1999) Nucleic Acids Res. 27:1912-18).

Fluorescent labels can be attached to DNA using standard procedures, e.g. for a review see Haugland, “Covalent Fluorescent Probes,” in Excited States of Biopolymers, Steiner, Ed. (Plenum Press, New York, 1983), incorporated by reference herein in its entirety. In a preferred embodiment of the invention, a fluorescent group can be covalently attached to a desired primer by reaction with a 5′-amino-modified oligonucleotide in the presence of sodium bicarbonate and dimethylformamide, as described in U.S. patent application Ser. No. 09/169,440. Alternatively, the reactive amine can be attached by means of the linking agents disclosed in U.S. Pat. No. 4,757,141. Alternatively, covalently tagged primers can be obtained commercially (e.g., from Midland Certified Reagent, Co.). Fluorescent dyes are available form Molecular Probes, Inc. (Eugene, Oreg.), Operon Technologies, Inc., (Alameda, Calif.) and Amersham Pharmacia Biotech (Piscataway, N.J.), or can be synthesized using standard techniques. Fluorescent labeling is described in U.S. Pat. No. 4,855,225.

In preferred embodiments of the invention, the chemical tag is a non-polar group. An example of a non-polar tag includes a hydrocarbon group, wherein the hydrocarbon group is selected from the group consisting of alkyl, cycloalkyl, aryl and arylalkyl groups. The number of carbon atoms in the hydrocarbon group can be up to about 18. Alkyl groups having up to 8 carbon atoms are preferred. In a preferred embodiment of the invention, the chemical tag is biotin.

Alternatively, the chemical tag can be a charged (i.e., ionic) group. In all instances, it is important that the chemical tag be capable of interacting with the separation medium. Preferably, the chemical tag should have a substantial affinity for the separation medium. In the case where the chemical tag is hydrophobic, this affinity can be achieved by using a separation medium with a non-polar surface.

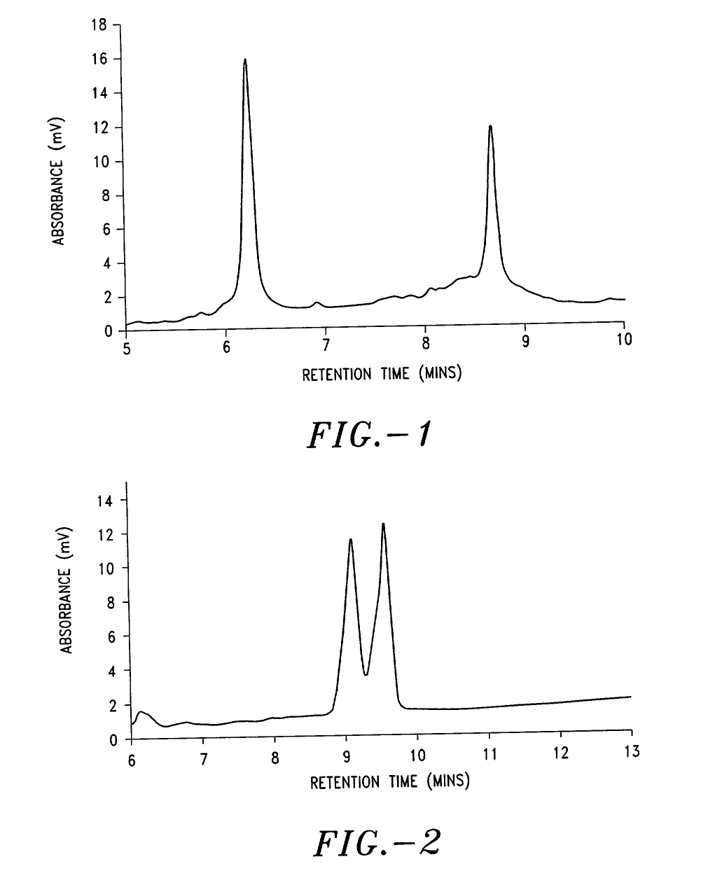

Under denaturing conditions, the ssDNA strands of a typical amplification product will typically migrate with very similar retention times, depending to some extent on the specific sequences. However, attaching a chemical tag with an affinity for the separation medium to one of the strands of a dsPCR product will cause an increased retention time. For example, attachment of a biotin tag will increase the hydrophobicity of the strand, which will increase the strand's affinity for a hydrophobic separation surface. For example, FIG. 1 shows the difference in IP-RP-HPLC retention times for two ssDNA strands, one non-biotinylated and the other biotinylated. The results demonstrate that under the conditions used, there is a two minute difference in the retention time of the ssDNA, compared with the biotinylated strand.

By incorporating one tagged (e.g., biotinylated) and one normal primer in a PCR, the separation of the ssDNA species by IP-RP-HPLC under denaturing conditions can be achieved.

Other features of the invention will become apparent in the course of the following descriptions of exemplary embodiments which are given for illustration of the invention and are not intended to be limiting thereof.

Procedures described in the past tense in the examples below have been carried out in the laboratory. Procedures described in the present tense have not yet been carried out in the laboratory, and are constructively reduced to practice with the filing of this application. All references referred to herein, including any patent, patent application or non-patent publication, are hereby incorporated by reference in their entirety.

### EXAMPLE 1

### Separation of Biotinylated and Non-biotinylated Oligodeoxynucleotides

A mixture of two oligodeoxynucleotides (“oligos”) of identical sequence (5′-GTAAAACGACGGCCAGT-3′; SEQ ID NO.: 1) was prepared, where one of the oligos was biotinylated at the 5′ end and the other non-biotinylated. 10 μL were separated by IP-RP-DHPLC on a DNASep chromatography cartridge (7.8 mm internal diameter and 50 mm length; Transgenomic, Inc.) using a WAVE Nucleic Acid Fragment Analysis System (Transgenomic, Inc., San Jose, Calif.). The stationary phase of the cartridge comprises a nonporous, C-18 alkylated poly(styrene-divinylbenzene) separation medium. The separation was conducted at 75° using the following gradient: Buffer A (0.1 M triethylammonium acetate (TEM), pH 7.0); Buffer B (0.1 M TEM, pH 7.0 containing 25% acetonitrile; starting at 20% Buffer B, the linear gradient was extended to 5% Buffer B over 14 minutes at a flow rate of 0.9 mL/min. The chromatogram (FIG. 1) shows that biotinylated oligo is eluted approximately two minutes later than the non-biotinylated oligo.

### EXAMPLE 2

### Chromatographic Separation of 200 Nucleotide ssDNA Fragments Amplified Using Asymmetrically Biotinylated Primers

PCRs were performed in a final volume of 50 μL using 100 ng of a plasmid template, pLitmus 28 (New England Bioloabs), 40 pmoles each of primers M1 and M2 (M1 5′-BGCGCAAGCTTAACAGCTATGACCATG (where B is biotin; SEQ ID NO.:2); M2 5′-GTAAAACGACGGCCAGT (SEQ ID NO.: 3)), 25 mM dNTPs and 1.25 U Taq polymerase (Promega). PCRs were carried out at 95° C. for 1 minute, followed by 50° C. for 1 minute and 72° C. for 1 minute for 30 cycles. At the end of the amplification reaction, 5 μL of the PCR product was injected directly onto the column (column and buffers are as described in Example 1) equilibrated at 75° C. using the following conditions: A linear gradient was entended from 30% to 70% buffer B over 10 minutes at a flow rate of 0.9 mL/min. The resulting chromatogram (FIG. 2) shows the presence of two peaks: the two ssDNA species. The biotinylated ssDNA is seen to have a longer retention time, although the resolution of the two strands is diminished relative to the oligos of Example 1. These results demonstrate that a 200 nucleotide ssDNA fragment can be separated from biotinylated ssDNA of the same length.

### EXAMPLE 3

### Chromatographic Separation of an Amplified Random DNA Pool Using Asymmetrically Biotinylated Primers

A series of PCRs were performed to amplify a random pool of DNA similar to that used for in vitro selection of ssDNA aptamers (Bock et al. (1992) Nature 355:564-66; Ellington and Szostak (1992) Nature 355:850-52). All PCRs were carried out in a final volume of 50 μL using 1 pmole of the following template ssDNA: 5′-ATACCAGCTTATTCAATT-(N50)-AGATAGTAAGTGCAATCT-3′ (SEQ ID NO.:4), and 40 pmoles each of primer R2: 5′-ATACCAGCTTATTCAATT-3′ (SEQ ID NO.: 5) and R3:5′-BAGATAGTAAGTGCAATCT-3′ (SEQ ID NO.: 6, where B represents biotin), 25 mM dNTPs, and 1.25 U Taq polymerase (Promega). The PCR conditions used were 95° C. for 1 minute, 50° C. for 1 minute and 72° C. for 1 minute for 15 cycles. At the end of the amplification phase a 5 μL fraction of a 50 μL PCR product was directly injected onto a DNASep column at 45° C. (non-denaturing conditions) and 75° C. (denaturing conditions) in two separate experiments. The sample was run using the following chromatography conditions: Buffer A (0.1 M triethylammonium acetate (TEM), pH 7.0); Buffer B (0.1 M TEM, pH 7.0 containing 25% acetonitrile; starting at 30% Buffer B, the linear gradient was extended to 70% Buffer B over 13 minutes at a flow rate of 0.9 mL/min.

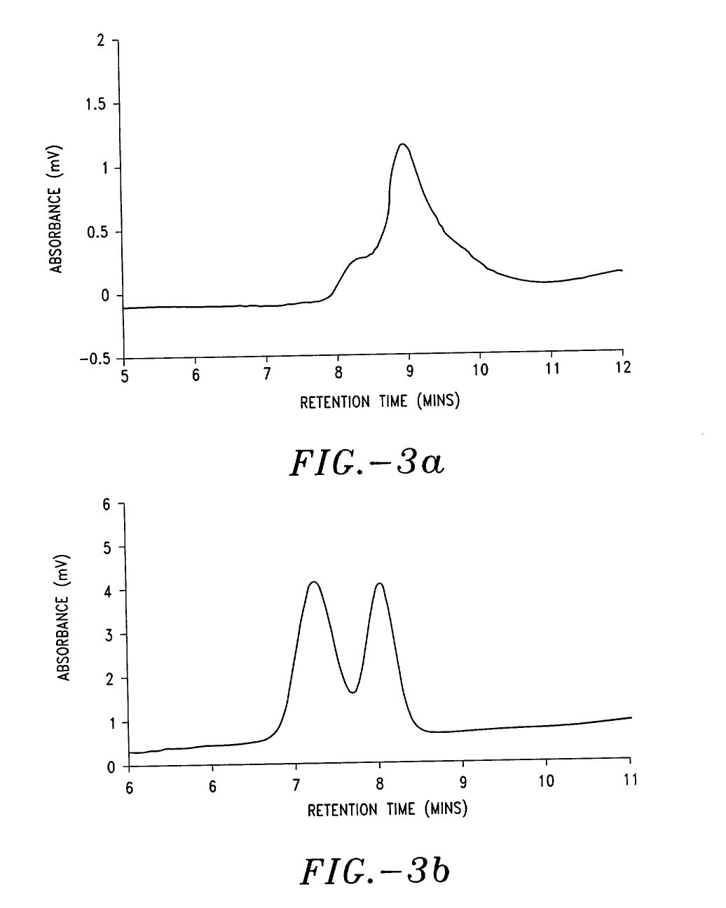

FIG. 3 shows the chromatograms obtained from the analysis of the dsPCR products run under non-denaturing conditions (FIG. 3a) and denaturing conditions (FIG. 3b). The results show that at 45° C. the dsDNA elutes as a single broad peak owing to, in this example, the random nature of the DNA pool (FIG. 3a). However, when the amplified DNA is separated under denaturing conditions (75° C.) the two ssDNA species are resolved, with a difference in retention time of approximately 0.75 minutes owing to the influence of the biotin moiety (FIG. 3b).

### EXAMPLE 4

### Separation of 50 Nucleotide Asymmetrically Labeled Fluorescent Duplex

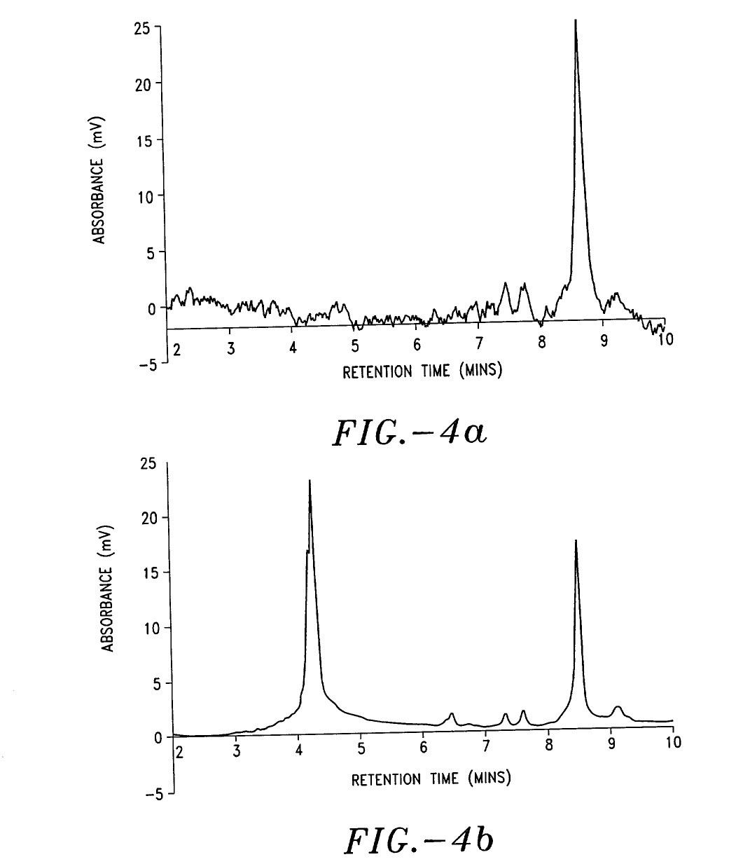

In this example a fluorescent tag (6-FAM) was used to isolate ssDNA by denaturing RP-IP-HPLC. The following complementary DNA strands were used, wherein each strand was 50 nucleotides in length, and one stand included a 5′-FAM group. (5′-FAM-TACCGACGTCATTCGCAGAGCATATAAGGTGAGGTAGGATAGCTACGTC (SEQ ID NO.: 6) and 5′-GACGTAGCTATCCTACCTCACCTTATATGCTCTGCGAATGACGTCGGTA (SEQ ID NO. 7)) The duplex DNA was injected onto the column (column and buffers are as described in Example 1) under denaturing conditions (75° C.) and the sample was run using the following conditions: a linear gradient from 40% to 70% buffer B over 12 minutes at a flow rate of 0.9 mL/min. The analysis was performed using both UV detection (260 nm) and fluorescence detection (Ex: 494 nm, Em: 525 nm). The resulting chromatograms (FIG. 4) show two peaks with UV detection (FIG. 4a); however, when the same sample is analyzed using fluorescence detection (FIG. 4b) only the fluorescent ssDNA is seen at the longer retention time. These results demonstrate that fluorescently labeled ssDNA can be rapidly isolated using denaturing DNA chromatography. Similar results were obtained using hexachlorofluorescein (HEX) and tetrachlorofluorescein (TET), demonstrating that ssDNA with different fluorescent tags can be isolated using this procedure. Fluorescent DNA can also be isolated from dsPCR products by means of a fluorescent primer in a system analogous to that used in the PCR with a biotinylated primer.

### EXAMPLE 5

### Separation of 200 bp Asymmetrically Labeled PCR Product

PCRs were performed in a final volume of of 50 μL using 100 ng of a plasmid template, pUC18 (New England Biolabs), 40 pmols of each primer (M3 5′-FGTAAAACGACGGCCAGT (where F=6-FAM; SEQ ID NO.: 7) and M4 5′-AACAGCTATGACCATG (SEQ ID NO.: 8)), 25 mM dNTPs and 1.25 U Taq polymerase. The PCRs were carried out at 95° C. for 1 minute, followed by 50° C. for 1 minute and 72° C. for 1 minute for 30 cycles. At the end of the amplification reaction, 5 UL of the PCR product was injected directly onto the column (same column and buffer as Example 1) equilibrated at 75° C. using the following conditions: a linear gradient from 30% to 70% buffer B over 10 minutes at a flow rate of 0.9 mL/min.

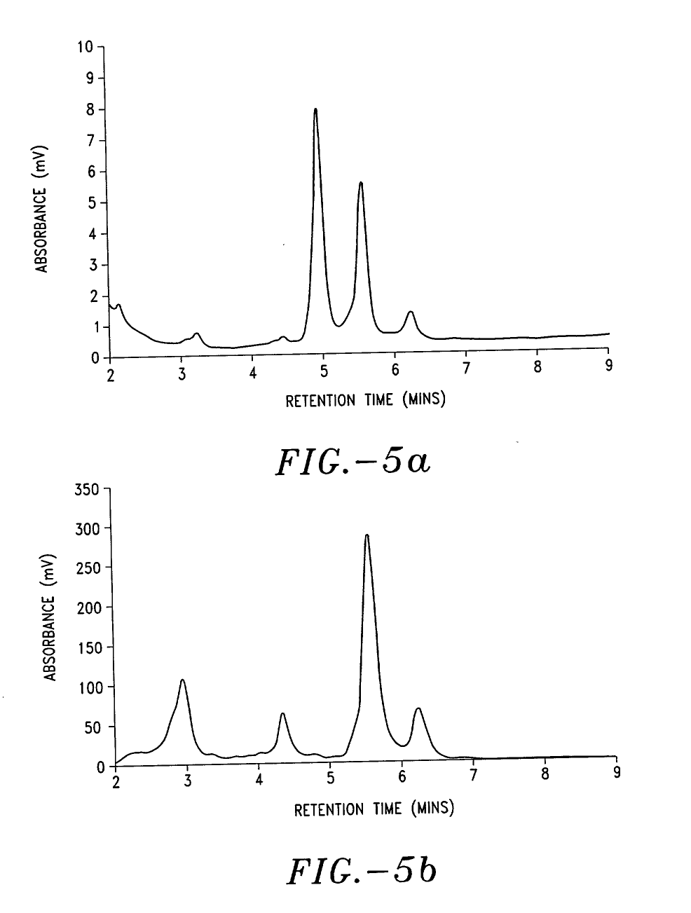

Analysis was performed as described in Example 3; the resulting chromatograms are shown in FIG. 5. The two single strands can clearly be seen using UV detection (FIG. 5a) and one main peak is seen using fluorescence detection (FIG. 5b). These results demonstrate the ability of this procedure to purify the fluorescent ssDNA from other PCR artefacts (e.g. primer dimers), which are eluted before the fluorescent ssDNA PCR product.

The invention claimed is:

1. A method for separating the complementary single-stranded polynucleotide products of an amplification reaction from one another comprising:
a) amplifying a target polynucleotide by means of two oligonucleotide primers, wherein one primer is capable of hybridizing to the target polynucleotide and the other primer is capable of hybridizing to the complement of the target polynucleotide, and wherein one of the primers comprises a chemical tag, thereby producing an amplification product mixture comprising a tagged amplification product of the target polynucleotide and a complementary non-tagged amplification product;
b) applying the amplification product mixture to a separation medium, wherein the chemical tag is capable of interacting with the separation medium; and
c) eluting the amplification products from the separation medium by means of a mobile phase under denaturing conditions, wherein the interaction between the tag and the separation medium results in the physical separation of the tagged amplification product from the non-tagged amplification product.

2. The method of claim 1, wherein at least one of the amplification products is detected.

3. The method of claim 1, wherein at least one of the amplification products is collected.

4. The method of claim 1, wherein the amplification product mixture is applied in the presence of a first counterion agent, wherein the separation medium has a non-polar surface, and wherein the mobile phase contains an organic solvent.

5. The method of claim 4, wherein the separation medium has a nonpolar separation surface that is substantially free of multivalent cations that are capable of interfering with polynucleotide separations.

6. The method of claim 5, wherein the solutions used are substantially free of multivalent cations capable of interfering with polynucleotide separations.

7. The method of claim 1, wherein the chemical tag has an affinity towards the separation medium.

8. The method of claim 7, wherein the chemical tag is hydrophobic.

9. The method of claim 7, wherein the chemical tag is biotin.

10. The method of claim 7, wherein the chemical tag is charged.

11. The method of claim 7, wherein the tag comprises a fluorescent group.

12. The method of claim 4, wherein the separation medium comprises polymer beads having an average diameter of 0.5 to 100 microns, the beads being unsubstituted polymer beads or polymer beads substituted with a moiety selected from the group consisting of hydrocarbon having from one to 1,000,000 carbons.

13. The method of claim 4, wherein the separation medium comprises particles selected from the group consisting of silica, silica carbide, silica nitrite, titanium oxide, aluminum oxide, zirconium oxide, carbon, insoluble polysaccharide, and diatomaceous earth, the particles having separation surfaces which are coated with a hydrocarbon or non-polar hydrocarbon substituted polymer, or have substantially all polar groups reacted with a non-polar hydrocarbon or substituted hydrocarbon group, wherein the surfaces are non-polar.

14. The method of claim 4, wherein the medium comprises a polymeric monolith.

15. The method of claim 4, wherein the medium comprises a derivatized silica gel monolith.

16. The method of claim 8, wherein the tag comprises a hydrocarbon group, wherein the hydrocarbon group is selected from the group consisting of alkyl, cycloalkyl, aryl and arylalkyl groups.

17. The method of claim 4, wherein the separation medium has been subjected to acid wash treatment to remove any residual surface metal contaminants.

18. The method of claim 4, wherein the separation medium has been subjected to treatment with a multivalent cation binding agent.

19. The method of claim 4, wherein the organic solvent is selected from the group consisting of alcohol, nitrile, dimethylformamide, tetrahydrofuran, ester, ether, and mixtures of one or more thereof.

20. The method of claim 19, wherein the organic solvent comprises acetonitrile.

21. The method of claim 4, wherein the mobile phase contains a second counterion agent, which may or may not be the same as the first counterion agent.

22. The method of claim 21, wherein the first and second counterion agents are selected from the group consisting of lower alkyl primary amine, lower alkyl secondary amine, lower alkyl tertiary amine, lower trialkylammonium salt, quaternary ammonium salt, and mixtures of one or more thereof.

23. The method of claim 22, wherein the first and second counterion agents are selected from the group consisting of octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyidiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, tetrabutylammonium acetate, triethylammonium hexafluoroisopropyl alcohol, and mixtures of one or more thereof.

24. The method of claim 23, wherein the counterion agent is tetrabutylammonium acetate.

25. The method of claim 23, wherein the counterion agent is triethylammonium acetate.

26. The method of claim 4, wherein amplification is achieved by PCR.

27. The method of claim 4, wherein the amplification products are DNA molecules.
